(19) United States
(12) Patent Application Publication
(10) Pub. No.: US 2013/0183658 A1
(43) Pub. Date: Jul. 18, 2013

(54) Methods and Devices for Rapid Detection of Live Microorganisms by Aptamers and/or Antibodies Immobilized on Permeable Membranes

(71) Applicant: Bret T. Barnhizer, Hubbard, OH (US)

(72) Inventor: Bret T. Barnhizer, Hubbard, OH (US)

(21) Appl. No.: 13/351,081

(22) Filed: Jan. 16, 2012

(51) Int. Cl. G01N 33/569; G01N 33/577; C12M 1/34; G01N 21/64

(52) U.S. Cl. 435/5; 435/7.1; 435/7.9; 435/287.2

(57) ABSTRACT

The present invention provides methods, devices and test kits for rapid detection and identification of one or more live target microorganisms in a liquid sample or grown on plates containing solid nutrient media. The invention includes mixing the one or more target microorganisms with one or more aptamers and/or one or more antibodies, each conjugated to a reporter compound and specific for a first site on the one or more target microorganisms to form a mixture. The mixture is placed on a permeable membrane having immobilized thereon one or more aptamers linked to an amine compound, and/or one or more antibodies, each specific for a second site on the one or more target microorganisms or a site on the aptamer conjugate and/or antibody conjugate. A detection solution is added to the membrane, and detection and identification of the one or more target microorganisms is achieved in less than one hour.

### FIELD OF THE INVENTION

The present invention relates generally to the field of microbiology and, in particular, to microbiological diagnostics for very rapid detection and identification of target microorganisms.

### BACKGROUND OF THE INVENTION

Modern microbiological diagnostic assays employ two different growth protocols for analysis, i.e., detection, identification and/or enumeration, of microorganisms: (1) analysis without preliminary growth of the microorganisms or (2) analysis after preliminary growth of microorganisms. Analysis of microorganisms without preliminary growth includes the use of methods such as: (1) immunological analyses, e.g., immunofluorescence, radioimmunoassay, enzyme immunoassay (EIA) for single cells and others; (2) DNA/RNA analyses via polymerase chain reaction (PCR); and (3) flow cytometry (FC) analyses (detection of single cells after labeling with fluorescent antibodies or fluorogenic substrates). Artificial substrates may also be used for detection and analysis of microorganisms by microscopic means. Microbiological diagnostic assays that analyze microorganisms after preliminary growth of the microorganisms include enzyme-linked immunosorbent assay (ELISA), mass-spectrometry, Fourier transform infrared (FTIR) spectroscopy, immune analyses and others.

Whether one chooses to use diagnostic assays with or without preliminary growth of microorganisms, all of the current diagnostic assays are cost, time and labor intensive and require the use of sophisticated laboratory equipment and personnel.

Along with the above-described sophisticated techniques, the traditional method of colony growth on a Petri plate still is the most common method used to detect live microorganisms in a sample. However, analysis by Petri plate also can be time and labor intensive. In order to create colonies, inoculated plates typically are incubated approximately 24 to 48 hours for bacteria and 72 to 120 hours for fungi. Thus, a relatively long time is needed to form colonies easily visible to the naked eye. If the sample arises from a time-sensitive biohazard incident, a hospital patient in critical condition, or industrial (food, pharmaceutical) products having a short shelf life, then time is of the essence and time-consuming incubation and serial testing can be a substantial burden with potentially life-threatening or profit loss consequences. Colonies appearing on solid nutrient media typically are counted for detection and enumeration of total microbial growth or are removed and analyzed according to traditional microbiological procedures, e.g., mass-spectrometry, Fourier transform infrared spectroscopy (FTIR) spectroscopy, chromatography, immunoassays or PCR.

In the field of medicine, in the food, biotechnological and pharmaceutical industries, for military and civilian defense, and environmental control, it is very important to have rapid, simple and reliable identification of colonies and microcolonies. For example, pathogens which affect the food industry are E. coli, Salmonella spp., Listeria spp., Pseudomonas aeruginosa, Staphylococcus aureus, some Lactobacillus spp., Bacillus cereus, some yeast and molds and other microorganisms. Pathogens which require rapid identification for military and civilian defense include, for example, anthrax, where time is of the essence. Reliable monoclonal and polyclonal antibodies exist for a majority of these organisms but they are most useful only after a high quantity of cells are grown on a special media or broth and then identified by EIA or ELISA using sophisticated and expensive equipment such as VITEC™ (bioMerieux) by fluorescence or using 96-well plates with specific antibodies immobilized inside the wells.

Consequently, there is a need for a rapid, accurate and cost-effective method to selectively detect and identify live microorganisms which overcomes the limitations of the prior art.

### SUMMARY OF THE INVENTION

The present invention fulfills this need by providing methods and devices for very rapid detection and identification of live target microorganisms contained in a liquid sample or after a short growth time of a sample on nutrient media. The detection and identification of target microorganisms may be achieved in one hour or much less, a very short time period not previously encountered with other diagnostic assays known to date.

In one aspect of the invention, there is provided a method for rapid detection and identification of one or more live target microorganisms in a sample, the method comprising the steps of obtaining a sample containing at least one target microorganism; forming a mixture containing the at least one microcolony from the at least one target microorganism and at least one first aptamer conjugated to a reporter compound and selected for a binding site of the at least one target microorganism and/or of at least one first antibody conjugated to a reporter compound and specific for an antigenic site of the at least one target microorganism; washing the mixture; adding the mixture to a permeable membrane, the permeable membrane having immobilized thereon at least one second aptamer selected for a binding site of the at least one first aptamer and/or at least one second antibody specific for an antigenic site of the at least one first antibody; washing any non-bound microorganisms from the permeable membrane; and adding a detection solution to the permeable membrane in order to detect and identify the at least one target microorganism, wherein the detection and identification of the at least one microorganism is achieved in less than one hour.

In an embodiment, the sample is a liquid sample containing at least one or more live target microorganisms.

In another embodiment, the sample is obtained by placing a sample containing at least one target microorganism on a nutrient medium and incubating the sample for a period of time in order to grow at least one microcolony from the at least one target microorganism.

In another aspect of the invention, there is provided a method for rapid detection and identification of one or more live target microorganisms in a sample, comprising the steps of obtaining a sample containing at least one target microorganism; forming a mixture containing the at least one microcolony from the at least one target microorganism and at least one first aptamer conjugated to a reporter compound and selected for a first binding site of the at least one target microorganism and/or at least one first antibody conjugated to a reporter compound and specific for a first antigenic site of the at least one target microorganism; adding the mixture to a permeable membrane having immobilized thereon at least one second aptamer selected for a second binding site of the at least one target microorganism and/or at least one second antibody specific for a second antigenic site of the at least one target microorganism; washing any non-bound microorganisms from the permeable membrane; and adding a detection solution to the permeable membrane in order to detect and identify the at least one target microorganism, wherein the detection and identification of the at least one microorganism is achieved in less than one hour.

In an embodiment, the sample is a liquid sample containing at least one or more live target microorganisms.

In another embodiment, the sample is placed on a nutrient medium and incubated for a period of time in order to grow at least one microcolony from the at least one target microorganism.

In another aspect of the invention, there is provided a device for rapid detection and identification of one or more live target microorganisms in a sample, the device comprising a container having therein nutrient medium to grow at least one microcolony from at least one live target microorganism; at least one first aptamer conjugated to a reporter compound and selected against a binding site of the at least one target microorganism and/or at least one first antibody conjugated to a reporter compound and specific for an antigenic site of the at least one target microorganism; a permeable membrane having immobilized thereon at least one second aptamer selected against a binding site of the at least one first aptamer and/or at least one second antibody specific for an antigenic site of the at least one first antibody; a portable washing apparatus; and a detection solution for detecting and identifying the at least one target microorganism bound on the permeable membrane in less than one hour.

In another aspect of the invention, there is provided a device for rapid detection and identification of one or more live target microorganisms in a sample, the device comprising a container having therein nutrient medium to grow at least one microcolony from at least one live target microorganism; at least one first aptamer conjugated to a reporter compound and selected against a first binding site of the at least one target microorganism and/or at least one first antibody conjugated to a reporter compound and specific for a first antigenic site of the at least one target microorganism; a permeable membrane having immobilized thereon at least one second aptamer selected against a second binding site of the at least one target microorganism and/or at least one second antibody specific for a second antigenic site of the at least one target microorganism; a portable washing apparatus; and a detection solution for detecting and identifying the at least one target microorganism bound on the permeable membrane in less than one hour.

The invention also includes test kits comprised of the methods and devices described hereinabove.

The at least one second aptamer of the invention may be linked to an amine compound, which includes without limitation, bovine serum albumin, amine linkers and other compounds which contain an amine group.

The at least one type of antibody includes, without limitation, a monoclonal or a polyclonal antibody.

Target microorganisms that can be detected and identified according to the embodiments of the present invention include, without limitation, bacteria, yeasts, fungi, viruses, cells and eukaryotic cells.

Suitable nutrient media contemplated for growing the at least one microorganism in a sample include, without limitation, agar such as tryptic soy agar or sheep blood agar.

Suitable permeable membranes contemplated for use in the present invention include, without limitation, polymers such as nitrocellulose, regenerated cellulose and its derivatives such as cellophane, cuprophane and dialysis membranes, nylon or polyvinylidene fluoride (PVDF) and other polymers such as organic or inorganic materials with naturally occurring pores or artificially created pores.

Suitable reporter compounds contemplated for use in the present invention include, without limitation, chromogenic substrates such as tetrazolium dyes such as MTT [3-(4,5-dimethylthiazol-2-yl)-2,5-diphenyltetrazolium bromide], INT [2-(p-iodophenyl-3-(p-nitrophenyl tetrazolium chloride], resazurine and other chromogenic substrates; fluorogenic substrates such as fluorescein isothiocyanate (FITC); and enzymes such as horse-radish peroxidase.

Suitable detection solutions contemplated for use in the present invention include, without limitation, enzymes such as alkaline phosphatase and staining solutions such as 3,3′-diaminobenzidine (DAB).

Portable washing apparatuses are well known in the art, any of which may be used in the methods and devices of the invention.

The methods and devices of the invention allow for rapid detection and identification of target microorganisms in one hour or less. In an embodiment, target microorganisms of the invention are detected and identified in 30 minutes or less.

### BRIEF DESCRIPTION OF THE DRAWINGS

The drawings included herewith are incorporated in and form a part of this specification. The drawings illustrate embodiments of the present disclosure and, together with the description, serve to explain the principles of the disclosure. It should be understood that the drawings referred to in this description are not drawn to scale unless specifically noted as such.

FIG. 1 shows an aptamer (referred to herein as a first aptamer) bound by another aptamer (referred to herein as a second aptamer). The first aptamer is conjugated to a reporter compound and is bound to a target microorganism. The second aptamer is immobilized on a permeable membrane via linkage to an amine compound.

FIG. 2 shows an aptamer (referred to herein as a first aptamer) bound to one site of a target microorganism, the first aptamer conjugated to a reporter compound, and a permeable membrane having an immobilized aptamer thereon (referred to herein as a second aptamer) bound to a second site of the target microorganism. The second aptamer is linked to an amine compound which immobilizes the second aptamer to the permeable membrane.

FIG. 3 shows an antibody (referred to herein as a first antibody) bound by another antibody (referred to herein as a second antibody). The first antibody is conjugated to a reporter compound and is bound to a target microorganism. The second antibody is immobilized on a permeable membrane.

FIG. 4 shows an antibody (referred to herein as a first antibody) bound to one site of a target microorganism, the first antibody conjugated to a reporter compound, and a permeable membrane having an immobilized antibody thereon (referred to herein as a second antibody), the second antibody bound to a second site of the target microorganism.

FIG. 5 shows an antibody (referred to herein as a first antibody) bound by an aptamer (referred to herein as a second aptamer), the first antibody conjugated to a reporter compound and bound to a target microorganism. The second aptamer is immobilized on a permeable membrane via linkage to an amine compound.

FIG. 6 shows an antibody (referred to herein as a first antibody) bound to one site of a target microorganism, the first antibody conjugated to a reporter compound, and a permeable membrane having an immobilized aptamer thereon (referred to herein as a second aptamer). The second aptamer is bound to a second site of the target microorganism and is immobilized on the permeable membrane via linkage to an amine compound.

FIG. 7 shows an aptamer (referred to herein as a first aptamer) bound by an antibody (referred to herein as a second antibody), the first aptamer conjugated to a reporter compound and bound to a target microorganism. The second antibody is immobilized on a permeable membrane.

FIG. 8 shows an aptamer (referred to herein as a first aptamer) bound to one site of a target microorganism, the first aptamer conjugated to a reporter compound, and a permeable membrane having an immobilized antibody thereon (referred to herein as a second antibody). The second antibody is bound to a second site of the target microorganism.

### DETAILED DESCRIPTION OF THE INVENTION

The present invention provides methods and devices for detecting and identifying live microorganisms which are extremely rapid and which do not require sophisticated laboratory equipment such as flow cytometers, mass-spectrometers, FTIR spectroscopy or PCR.

The present invention is based on the ability of live target microorganisms to bind selectively and with high specificity to unbound aptamers and/or antibodies and to aptamers and/or antibodies immobilized on permeable membranes.

The present invention allows for the detection and identification of many different types of microorganisms, such as bacteria, yeasts, fungi, spores, eukaryotic cells, cells, viruses or proteins.

Aptamers, nucleic acid sequences having unique properties, were first identified from very large random sequence oligonucleotide libraries in 1990 by Tuerk C. et al. (Science, 1990, 249:505-10) and Ellington, A. D. et al. (Nature, 1990; 346:818-22), both papers incorporated herein by reference. The selection method for aptamers, called the SELEX (systemic evolution of ligands by exponential enrichment) process, is a technique which screens very large combinatorial libraries of oligonucleotides by an iterative process of in vitro selection and amplification. Combinatorial libraries based on replicable biopolymers such as nucleic acids (and peptides) offer the convenience of iterative amplification of their members, making the screening process fast and easy.

The screening process, a random sequence oligonucleotide library is incubated with a target of interest. During this step, a very small fraction of individual sequences tends to interact with the target, and these sequences are separated from the rest of the library by means of any one of several physical separation techniques known in the art. Typically, nitrocellulose filter partitioning is used with protein targets that are retained on nitrocellulose. Small molecular targets are generally immobilized on a solid support to generate an affinity matrix, in which case sequences that do not interact with the target on the solid support can be removed easily by a simple washing step. The population of sequences bound to the target is isolated and amplified to obtain an enriched library to be used for the next selection/amplification cycle. The enrichment efficiency of high-affinity binders is governed by the stringency of selection at each round. The progress of the enrichment of high-affinity binders can be determined by carrying out binding analysis of enriching populations against the target. Once affinity saturation is achieved after several rounds of selection/amplification, the enriched library is cloned and sequenced to obtain the sequence information of each member. Individual sequences are further characterized on the basis of their ability to bind to the target. The majority of individual sequences, greater than 90%, in an enriched library are aptamers that bind successfully to the target used for selection.

Aptamers have the capacity to recognize virtually any class of target compounds with high affinity and specificity which rival antibodies in both therapeutic and diagnostic applications. Aptamers are much smaller and less complex than antibodies, and thus are easier to manufacture and modify. Aptamers mimic properties of antibodies in a variety of diagnostic formats, but are very different from antibodies, having characteristics that overcome many shortcomings of antibodies when used in diagnostic applications. Some of the shortcomings of antibodies include difficulty in growing high yields of monoclonal antibodies in vivo, as they typically are grown as hybridomas in peritoneal cavities of animals and purified from ascites fluid; performance of the same antibody tends to vary from batch to batch, requiring immunoassays to be re-optimized with each new batch of antibodies; identification of antibodies that could recognize targets under conditions other than physiological conditions is not feasible; kinetic parameters of antibody-target interactions cannot be changed on demand; antibodies are sensitive to temperature and undergo irreversible denaturation; and antibodies have a limited shelf life.

In contrast, aptamers possess many advantages when used in diagnostic applications. First, aptamers exhibit extraordinary specificity. For example, the aptamer selected against theophylline, a purine derivative used to treat asthma, binds with a 10.000-fold lower affinity to caffeine, another purine analog, which differs from theophylline by only one methyl group. Second, aptamers can be identified through an in vitro process that does not depend on animals, cells, or in vivo conditions. As a result, the properties of aptamers can be modified on demand. Third, selection conditions can be manipulated to obtain aptamers with properties desirable for in vitro diagnostics. For example, aptamers that bind to a target in a non-physiological buffer and at non-physiological temperatures can be identified. Similarly, kinetic parameters, such as the on- and off-rates of aptamers, can be changed on demand. Fourth, because animals or cells are not involved in aptamer identification, toxins as well as compounds which do not elicit good immune responses can be used to generate high-affinity aptamers. Fifth, aptamers are produced by chemical synthesis with extreme accuracy and reproducibility. They are purified under denaturing conditions to a very high degree of purity. Therefore, little or no batch-to-batch variation is encountered in aptamer production. Sixth, reporter compounds such as biotin, HRP and fluorescein can be attached to aptamers at precise locations identified by the user. Functional groups that allow subsequent derivatization of aptamers with other compounds can also be attached during chemical synthesis of aptamers. Seventh, although aptamers undergo denaturation, the process is reversible. After being denatured, functional aptamers can be regenerated easily within minutes. Eighth, aptamers are stable to long-term storage and can be transported at ambient temperature, and thus can tolerate transportation without special requirements for cooling, eliminating the need for a continuous cold chain.

The present invention takes advantage of the superior qualities and unique characteristics of aptamers in concert with antibody technology to arrive at extremely rapid assays to detect and identify target microorganisms in less than one hour. In an embodiment, detection and identification is achieved in less than 30 minutes. In another embodiment, detection and identification is achieved in about 10 minutes.

The permeable membranes encompassed by the present invention include, without limitation, polymers such as nitrocellulose, regenerated cellulose and its derivatives such as cellophane, cuprophane and dialysis membranes, nylon or polyvinylidene fluoride (PVDF) and other polymers such as organic or inorganic materials with naturally occurring pores or artificially created pores. In particular, nitrocellulose membranes are able to bind amine (NH₂)-containing compounds on their surfaces because of the strong interaction of nitrite (NO₂) groups contained in the nitrocellulose membrane with the NH₂ groups contained in the compounds. The aptamer compounds encompassed by the present invention are linked to NH₂-containing compounds which include, without limitation, amine linkers, biotin, bovine serum albumin (BSA) compounds or other NH₂-containing compounds, and thus are easily immobilized on the permeable membranes of the present invention. The NH₂-containing compounds are linked to the 5′-end of the aptamer compounds during oligonucleotide synthesis, according to synthetic protocols known by those in the art. All antibody compounds contain NH₂ groups, and thus the antibodies encompassed by the present invention also are easily immobilized on permeable membranes. Monoclonal antibodies, polyclonal antibodies or a combination of both may be used in the methods of the present invention. Lectins or other natural agglutinins also may be used in place of antibodies. Whether the permeable membrane of the invention has immobilized aptamers and/or antibodies on its surface, spaces in the permeable membrane not filled with the aptamers and/or antibodies are filled with a blocking buffer compound, such as milk powder, BSA, biotin or other proteinaceous compounds, so that all spaces of the permeable membrane are filled.

The following figures show the various configurations in which aptamers and/or antibodies may be employed in the methods of the invention to allow for the extraordinary and surprisingly rapid detection and identification of target microorganisms.

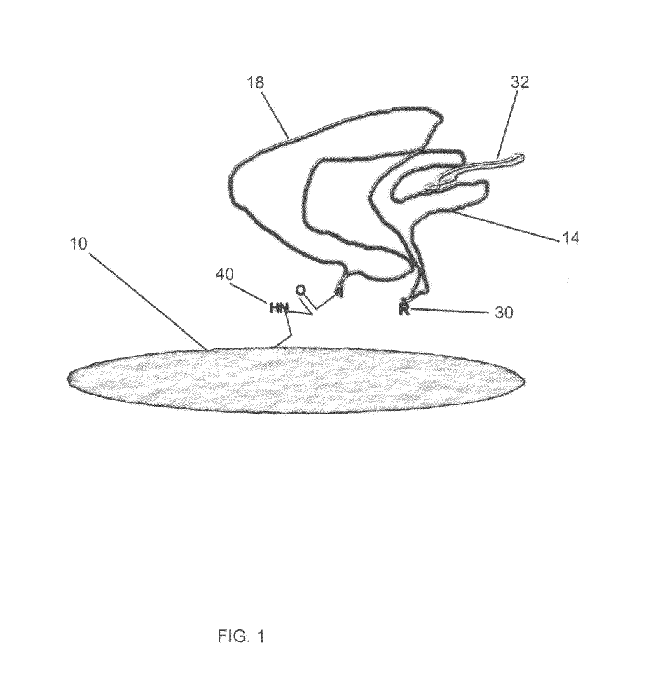

Referring to FIG. 1, a permeable membrane 10 is shown with an aptamer 18 (referred to herein as a second aptamer) immobilized thereon. The second aptamer 18 is linked to an amine compound 40 which immobilizes the second aptamer 18 to the permeable membrane 10. The second aptamer 18 is bound to a different aptamer 14 (referred to herein as a first aptamer), which is bound to a target microorganism 32. The first aptamer 14 is conjugated to a reporter compound 30 which includes, without limitation, chromogenic substrates such as tetrazolium dyes such as MTT [3-(4,5-dimethylthiazol-2-yl)-2,5-diphenyltetrazolium bromide], INT [2-(p-iodophenyl-3-(p-nitrophenyl tetrazolium chloride], resazurine and other chromogenic substrates; fluorogenic substrates such as fluorescein isothiocyanate (FITC); or enzymes such as horse-radish peroxidase. After the permeable membrane 10 is washed, a detection solution, which includes, without limitation, 3,3′-diaminobenzidine (DAB) or alkaline phosphatase, is added to the permeable membrane 10 which changes the color of the permeable membrane 10 in locations where the target microorganism 32 is attached to the permeable membrane 10 via binding to the immobilized second aptamer 18.

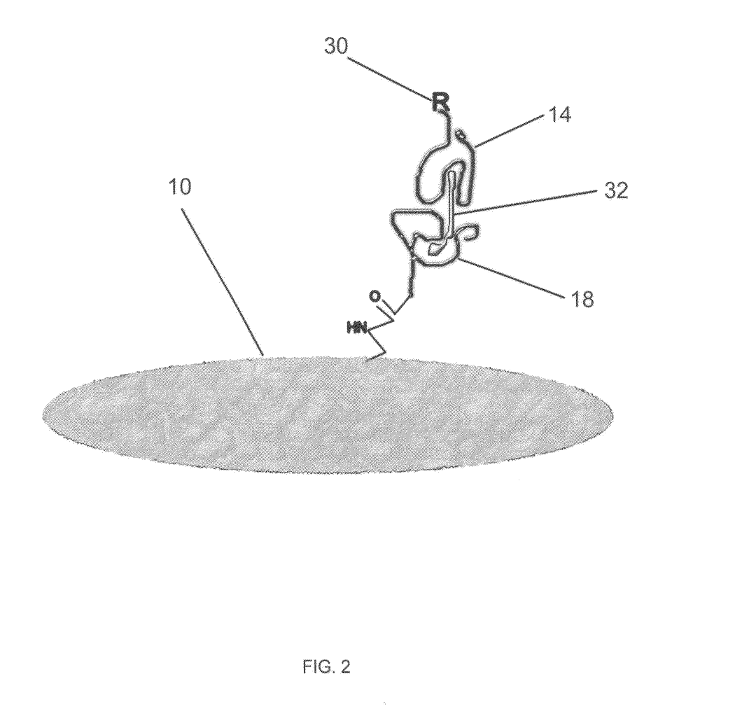

Referring to FIG. 2, a permeable membrane 10 is shown with an aptamer 18 (referred to herein as a second aptamer) immobilized thereon. The second aptamer 18 is linked to an amine compound which immobilizes the second aptamer 18 to the permeable membrane 10. The second aptamer 18 is bound to one site of a target microorganism 32 and a different aptamer 14 (referred to herein as a first aptamer) is bound to a different site of the target microorganism 32. The first aptamer 14 is conjugated to a reporter compound 30 as described above. After the permeable membrane 10 is washed, a detection solution, as described above, is added to the permeable membrane 10 which changes the color of the permeable membrane 10 in locations where the target microorganism 32 is attached to the permeable membrane 10 via binding to the immobilized second aptamer 18.

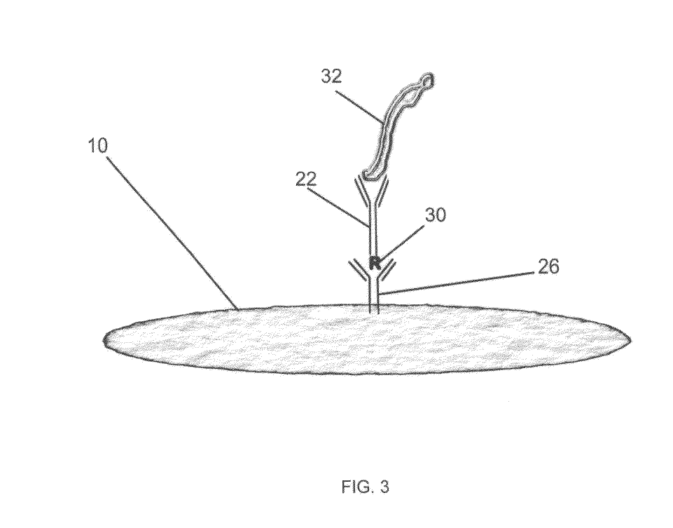

Referring to FIG. 3, a permeable membrane 10 is shown with an antibody 26 (referred to herein as a second antibody) immobilized thereon. The second antibody 26 is bound to a different antibody 22 (referred to herein as a first antibody), which is bound to a target microorganism 32. The first antibody 22 is conjugated to a reporter compound 30 as described above. After the permeable membrane 10 is washed, a detection solution, as described above, is added to the permeable membrane 10 which changes the color of the permeable membrane 10 in locations where the target microorganism 32 is attached to the permeable membrane 10 via binding to the immobilized second antibody 26.

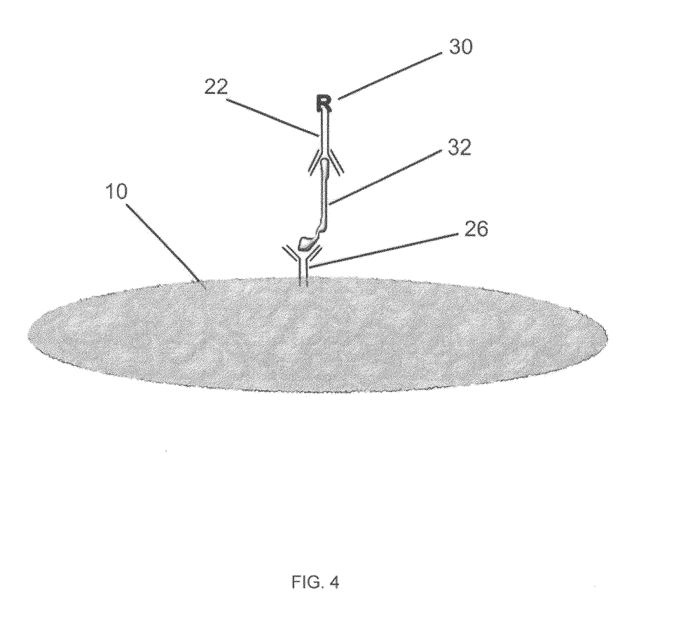

Referring to FIG. 4, a permeable membrane 10 is shown with an antibody 26 (referred to herein as a second antibody) immobilized thereon. The second antibody 26 is bound to an antigenic site of a target microorganism 32 and a different antibody 22 (referred to herein as a first antibody) is bound to a second antigenic site of the target microorganism 32. The first antibody 22 is conjugated to a reporter compound 30 as described above. After the permeable membrane 10 is washed, a detection solution, as described above, is added to the permeable membrane 10 which changes the color of the permeable membrane 10 in locations where the target microorganism 32 is attached to the permeable membrane 10 via binding to the immobilized antibody 26.

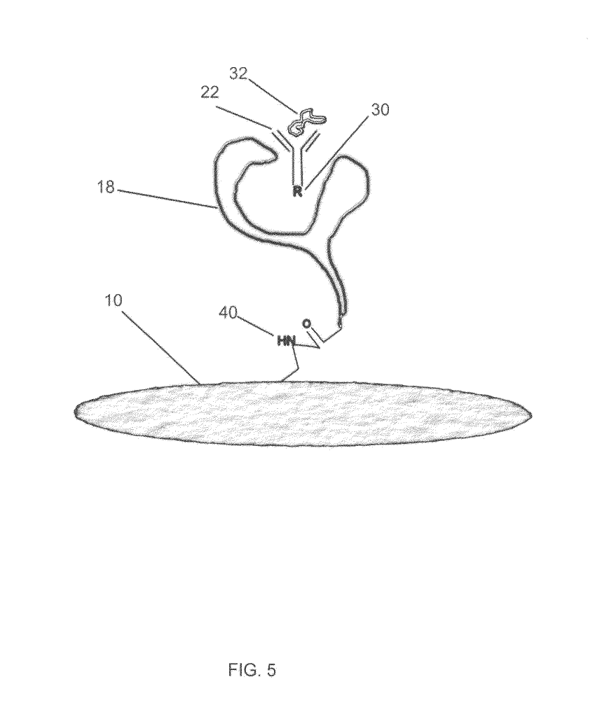

Referring to FIG. 5, a permeable membrane 10 is shown with an aptamer 18 (referred to herein as a second aptamer) immobilized thereon. The aptamer 18 is linked to an amine compound 40 which immobilizes the second aptamer 18 to the permeable membrane 10. The second aptamer 18 is bound to an antibody 22 (referred to herein as a first antibody), which is bound to a target microorganism 32. The first antibody 22 is conjugated to a reporter compound as described above. After the permeable membrane 10 is washed, a detection solution, as described above, is added to the permeable membrane 10 which changes the color of the permeable membrane 10 in locations where the target microorganism 32 is attached to the permeable membrane 10 via binding to the immobilized second aptamer 18.

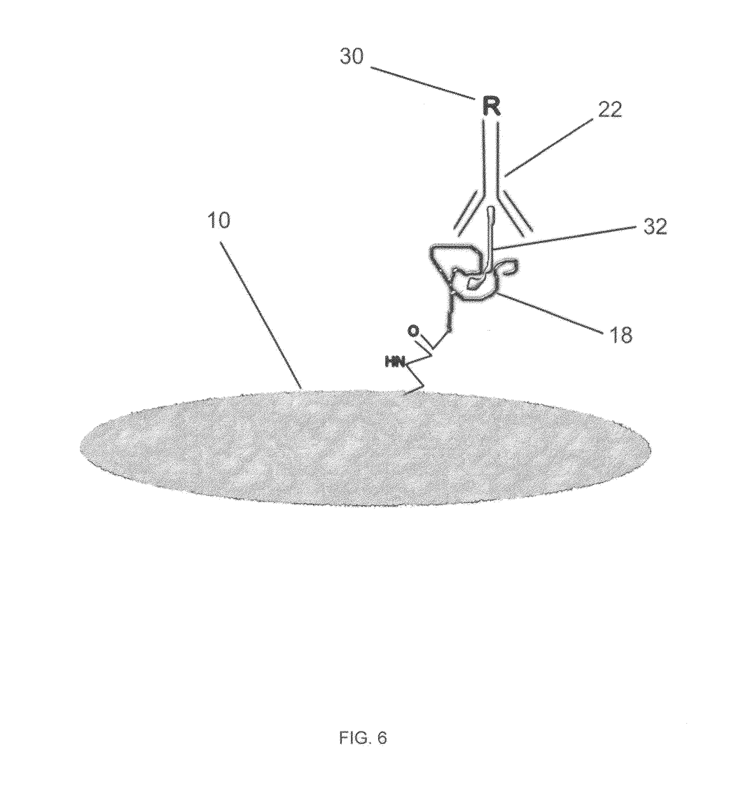

Referring to FIG. 6, a permeable membrane 10 is shown with an aptamer 18 (referred to herein as a second aptamer) immobilized thereon. The second aptamer 18 is linked to an amine compound which immobilizes the second aptamer 18 to the permeable membrane 10. The second aptamer 18 is bound to one site of a target microorganism 32 and an antibody 22 (referred to herein as a first antibody) is bound to a different antigenic site of the target microorganism 32. The first antibody 22 is conjugated to a reporter compound 30 as described above. After the permeable membrane 10 is washed, a detection solution, as described above, is added to the permeable membrane 10 which changes the color of the permeable membrane 10 in locations where the target microorganism 32 is attached to the permeable membrane 10 via binding to the immobilized second aptamer 18.

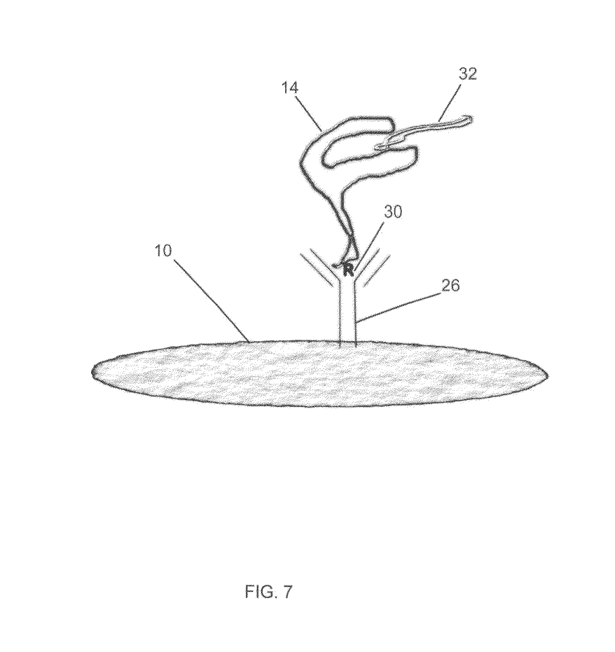

Referring to FIG. 7, a permeable membrane 10 is shown with an antibody 26 (referred to herein as a second antibody) immobilized thereon. The second antibody 26 is bound to an aptamer 14 (referred to herein as a first aptamer), which is bound to a target microorganism 32. The first aptamer 14 is conjugated to a reporter compound as described above. After the permeable membrane 10 is washed, a detection solution, as described above, is added to the permeable membrane 10 which changes the color of the permeable membrane 10 in locations where the target microorganism 32 is attached to the permeable membrane 10 via binding to the immobilized second antibody 26.

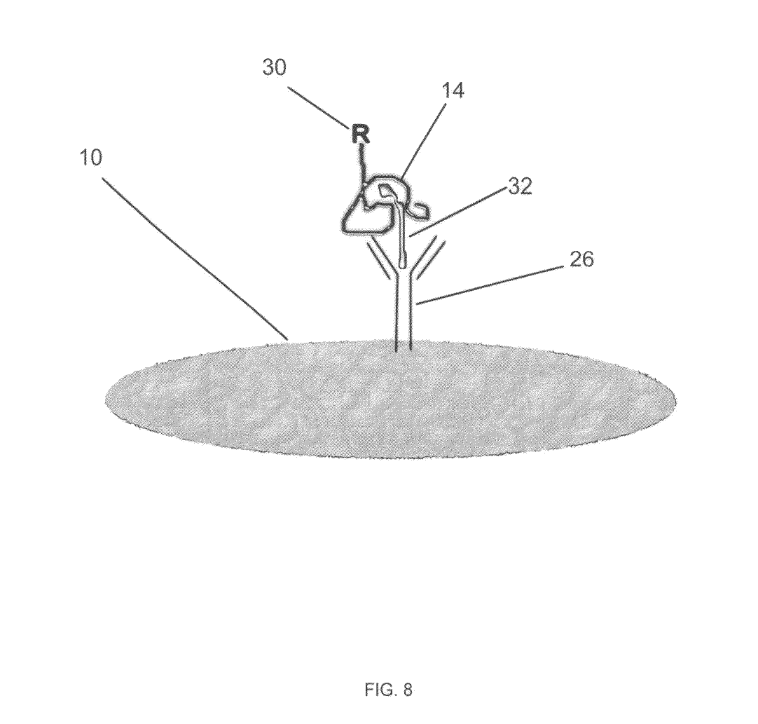

Referring to FIG. 8, a permeable membrane 10 is shown with an antibody 26 (referred to herein as a second antibody) immobilized thereon. The second antibody 26 is bound to an antigenic site of a target microorganism 32 and an aptamer 14 (referred to herein as a first aptamer) is bound to a different site of the target microorganism 32. The first aptamer 14 is conjugated to a reporter compound 30 as described above. After the permeable membrane 10 is washed, a detection solution, as described above, is added to the membrane 10 which changes the color of the permeable membrane 10 in locations where the target microorganism 32 is attached to the permeable membrane 10 via binding to the immobilized second antibody 26.

The present invention encompasses not only the foregoing described configurations of aptamers and/or antibodies, but also encompasses having more than one type of aptamer and/or more than one type of antibody immobilized on the permeable membrane and more than one type of aptamer and/or more than one type of antibody conjugated to a reporter compound, in order to allow for rapid detection and identification of more than one target microorganism.

### EXAMPLES

The present invention is more particularly described in the following non-limiting examples, which are intended to be illustrative only, as numerous modifications and variations therein will be apparent to those skilled in the art.

### Example 1

### Rapid Detection and Identification of Group B Streptococcus (GBS) Using Aptamers as Reporter and Capture Compounds

A liquid sample containing an unknown number of live bacterial species including the target bacterium GBS (also known as Streptococcus agalactiae) is set up as described below. Alternatively, a sample containing an unknown mixture of live bacterial species including the target bacterium GBS is poured on a Petri plate filled with sheep blood agar (SBA); the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of horse-radish peroxidase (HRP) conjugated to aptamers selected against GBS is added to bring the final volume up to 100 ml. The sample is spun at 3,000 rpm in a microcentrifuge and washed with phosphate buffered saline (PBS) three times. The pellet is resuspended in PBS. Twenty ml of the resuspended sample is added to a nitrocellulose permeable membrane which has been blocked overnight with 1% bovine serum albumin blocking compounds and containing immobilized aptamers (linked via amine compounds to the membrane) selected to bind to the aptamers conjugated with HRP. The permeable membrane then is washed three times. A drop of 3,3′-diaminobenzidine (DAB) (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where GBS is attached to the membrane via binding to the immobilized aptamers. GBS then is detected and identified on the permeable membrane due to the color change of the GBS microcolony(ies), which is distinguished from the color of any other species of bacteria contained in the mixture. GBS is detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 2

### Rapid Detection and Identification of GBS Using Aptamers as Reporter and Capture Compounds and Selected Against Two Different Sites of GBS

A liquid sample containing an unknown number of live bacterial species including the target bacterium GBS is set up as described below. Alternatively, a sample containing an unknown mixture of live bacterial species including the target bacterium GBS is poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to aptamers selected against one antigenic site of GBS is added to bring the final volume up to 20 ml. The 20 ml sample is added to a nitrocellulose permeable membrane which has been blocked overnight with 1% BSA blocking compounds and containing immobilized aptamers (linked via amine compounds to the membrane) selected to bind to a second antigenic site of GBS. The permeable membrane then is washed in a washing solution (PBS, pH 7.4 with 0.1% Tween 20) using a portable washing device at medium speed of rotation for about two minutes. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where GBS is attached to the membrane via binding to the immobilized aptamers. GBS then is detected and identified on the permeable membrane due to the color change of the GBS microcolony(ies), which is distinguished from the color of any other species of bacteria contained in the mixture. GBS is detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 3

### Rapid Detection and Identification of GBS Using Antibodies as Reporter and Capture Compounds

A liquid sample containing an unknown number of live bacterial species including the target bacterium GBS is set up as described below. Alternatively, a sample containing an unknown mixture of live bacterial species including the target bacterium GBS is poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to rabbit polyclonal antibodies against GBS is added to bring the final volume up to 100 ml. The sample is spun at 3,000 rpm in a microcentrifuge and washed with PBS three times. The pellet is resuspended in PBS. Twenty ml of the resuspended sample is added to a nitrocellulose permeable membrane which has been coated with 20 ml of a 1:40 dilution of mouse monoclonal antibody against rabbit IgG and blocked overnight with 1% BSA blocking compounds. The permeable membrane then is washed three times. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where GBS is attached to the membrane via binding to the immobilized antibodies. GBS then is detected and identified on the permeable membrane due to the color change of the GBS microcolony(ies), which is distinguished from the color of any other species of bacteria contained in the mixture. GBS is detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 4

### Rapid Detection and Identification of GBS Using Antibodies as Reporter and Capture Compounds and Selected Against Two Different Sites of GBS

A liquid sample containing an unknown number of live bacterial species including the target bacterium GBS is set up as described below. Alternatively, a sample containing an unknown mixture of live bacterial species including the target bacterium GBS is poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to rabbit polyclonal antibodies specific for an antigenic site of GBS is added to bring the final volume up to 20 ml. The 20 ml sample is added to a nitrocellulose permeable membrane which has been coated with 20 ml of a 1:40 dilution of rabbit polyclonal antibodies specific for a different antigenic site of GBS and blocked overnight with 1% BSA blocking compounds. The permeable membrane then is washed in a washing solution (PBS, pH 7.4 with 0.1% Tween 20) using a portable washing device at medium speed of rotation for about two minutes. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where GBS is attached to the membrane via binding to the immobilized aptamers. GBS then is detected and identified on the permeable membrane due to the color change of the GBS microcolony(ies), which is distinguished from the color of any other species of bacteria contained in the mixture. GBS is detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 5

### Rapid Detection and Identification of Bacillus anthracia (Anthrax) Using Aptamers as Reporter and Capture Compounds

A liquid sample containing an unknown number of live bacterial species including the target bacterium anthrax is set up as described below. Alternatively, a sample containing an unknown mixture of live bacterial species including the target bacterium anthrax is poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to aptamers selected against anthrax is added to bring the final volume up to 100 ml. The sample is spun at 3,000 rpm in a microcentrifuge and washed with PBS three times. The pellet is resuspended in PBS. Twenty ml of the resuspended sample is added to a nitrocellulose permeable membrane which has been blocked overnight with 1% BSA blocking compounds and containing immobilized aptamers (linked to amine linker compounds) selected to bind to the aptamers conjugated with HRP. The permeable membrane then is washed three times. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where anthrax is attached to the membrane via binding to the immobilized aptamers. Anthrax then is detected and identified on the permeable membrane due to the color change of the anthrax microcolony(ies), which is distinguished from the color of any other species of bacteria contained in the mixture. Anthrax is detected reliably at 10⁵ to 10² bacteria/ml in as little as 10 minutes, with minimal background observed, and with no cross-reactivity to other species of bacteria. This detection and identification assay is significantly more rapid than any other microbiological diagnostic assay known to date.

### Example 6

### Rapid Detection and Identification of Anthrax Using Aptamers as Reporter and Capture Compounds and Selected Against Two Different Sites of Bacillus anthracia

A liquid sample containing an unknown number of live bacterial species including the target bacterium anthrax is set up as described below. Alternatively, a sample containing an unknown mixture of live bacterial species including the target bacterium anthrax is poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to aptamers selected against one antigenic site of anthrax is added to bring the final volume up to 20 ml. The 20 ml sample is added to a nitrocellulose permeable membrane which has been blocked overnight with 1% BSA blocking compounds and containing immobilized aptamers (linked via amine compounds to the membrane) selected to bind to a second antigenic site of anthrax. The permeable membrane then is washed in a washing solution (PBS, pH 7.4 with 0.1% Tween 20) using a portable washing device at medium speed of rotation for about two minutes. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where anthrax is attached to the membrane via binding to the immobilized aptamers. Anthrax then is detected and identified on the permeable membrane due to the color change of the anthrax microcolony(ies), which is distinguished from the color of any other species of bacteria contained in the mixture. Anthrax is detected reliably at 10⁵ to 10² bacteria/ml in as little as 10 minutes, with minimal background observed, and with no cross-reactivity to other species of bacteria. This detection and identification assay is significantly more rapid than any other microbiological diagnostic assay known to date.

### Example 7

### Rapid Detection and Identification of Anthrax Using Antibodies as Reporter and Capture Compounds

A liquid sample containing an unknown number of live bacterial species including the target bacterium anthrax is set up as described below. Alternatively, a sample containing an unknown mixture of live bacterial species including the target bacterium GBS is poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to rabbit polyclonal antibodies against anthrax is added to bring the final volume up to 100 ml. The sample is spun at 3,000 rpm in a microcentrifuge and washed with PBS three times. The pellet is resuspended in PBS. Twenty ml of the resuspended sample is added to a nitrocellulose permeable membrane which has been coated with 20 ml of a 1:40 dilution of mouse monoclonal antibody against rabbit IgG and blocked overnight with 1% BSA blocking compounds. The permeable membrane then is washed three times. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where anthrax is attached to the membrane via binding to the immobilized antibodies. Anthrax then is detected and identified on the permeable membrane due to the color change of the anthrax microcolony(ies), which is distinguished from the color of any other species of bacteria contained in the mixture. Anthrax is detected reliably at 10⁵ to 10² bacteria/ml in as little as 10 minutes, with minimal background observed, and with no cross-reactivity to other species of bacteria. This detection and identification assay is significantly more rapid than any other microbiological diagnostic assay known to date.

### Example 8

### Rapid Detection and Identification of Anthrax Using Antibodies as Reporter and Capture Compounds and Selected Against Two Different Sites of Anthrax

A liquid sample containing an unknown number of live bacterial species including the target bacterium anthrax is set up as described below. Alternatively, a sample containing an unknown mixture of live bacterial species including the target bacterium anthrax is poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to rabbit polyclonal antibodies specific for an antigenic site of anthrax is added to bring the final volume up to 20 ml. The 20 ml sample is added to a nitrocellulose permeable membrane which has been coated with 20 ml of a 1:40 dilution of rabbit polyclonal antibodies specific for a different antigenic site of anthrax and blocked overnight with 1% BSA blocking compounds. The permeable membrane then is washed in a washing solution (PBS, pH 7.4 with 0.1% Tween 20) using a portable washing device at medium speed of rotation for about two minutes. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where anthrax is attached to the membrane via binding to the immobilized antibodies. Anthrax then is detected and identified on the permeable membrane due to the color change of the anthrax microcolony(ies), which is distinguished from the color of any other species of bacteria contained in the mixture. Anthrax is detected reliably at 10⁵ to 10² bacteria/ml in as little as 10 minutes, with minimal background observed, and with no cross-reactivity to other species of bacteria. This detection and identification assay is significantly more rapid than any other microbiological diagnostic assay known to date.

### Example 9

### Rapid Detection and Identification of Escherichia coli O157 H:7 (E. coli) and Salmonella Using Aptamers as Reporter and Capture Compounds

A liquid sample containing an unknown number of live bacterial species including the two target bacteria E. coli and Salmonella is set up as described below. Alternatively, a sample containing an unknown mixture of live bacterial species including the two target bacteria E. coli and Salmonella are poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to aptamers selected against E. coli and aptamers selected against Salmonella is added to bring the final volume up to 100 ml. The sample is spun at 3,000 rpm in a microcentrifuge and washed with PBS three times. The pellet is resuspended in PBS. Twenty ml of the resuspended sample is added to a nitrocellulose permeable membrane which has been blocked overnight with 1% BSA blocking compounds and containing immobilized thereon two different aptamers (each linked via amine compounds to the membrane). The first aptamer is selected to bind to the aptamers conjugated with HRP and selected against E. coli and the second aptamer is selected to bind to the aptamers conjugated with HRP and selected against Salmonella. The two different aptamers are localized on the permeable membrane in distinct locations to distinguish between the two species of bacteria. The permeable membrane then is washed three times. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where E. coli and Salmonella are attached to the membrane via binding to their respective immobilized aptamers. E. coli and Salmonella then are detected and identified on the permeable membrane due to the color change of the E. coli and Salmonella on the permeable membrane and their respective locations on the permeable membrane, which is distinguished from the color of any other species of bacteria which may be contained in the mixture. E. coli and Salmonella are detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 10

### Rapid Detection and Identification of E. coli and Salmonella Using Aptamers as Reporter and Capture Compounds and Selected Against Two Different Sites of E. coli and Salmonella

A liquid sample containing an unknown number of live bacterial species including the two target bacteria E. coli and Salmonella is set up as described below. Alternatively, a sample containing an unknown mixture of live bacterial species including the two target bacteria E. coli and Salmonella are poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to two different aptamers, one aptamer selected for a first antigenic site of E. coli and the other aptamer selected for a first antigenic site of Salmonella, is added to bring the final volume up to 20 ml. The 20 ml sample is added to a nitrocellulose permeable membrane which has been blocked overnight with 1% BSA blocking compounds and which has been coated with 20 ml of a 1:40 dilution of two aptamers (each linked via an amine compound to the membrane), the first aptamer selected for a second antigenic site of E. coli and the second aptamer selected for a second antigenic site of Salmonella. The permeable membrane then is washed in a washing solution (PBS, pH 7.4 with 0.1% Tween 20) using a portable washing device at medium speed of rotation for about two minutes. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where E. coli and Salmonella are attached to the membrane via binding to their respective immobilized aptamers. E. coli and Salmonella then are detected and identified on the permeable membrane due to the color change of the E. coli and Salmonella on the permeable membrane and their respective locations on the permeable membrane, which is distinguished from the color of any other species of bacteria which may be contained in the mixture. E. coli and Salmonella are detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 11

### Rapid Detection and Identification of E. coli and Salmonella Using Antibodies as Reporter and Capture Compounds

A liquid sample containing an unknown number of live bacterial species including the two target bacteria E. coli and Salmonella is set up as described below. Alternatively, a sample containing an unknown mixture of live bacterial species including the two target bacteria E. coli and Salmonella are poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to antibodies specific for an antigenic site of E. coli and antibodies specific for an antigenic site of Salmonella is added to bring the final volume up to 100 ml. The sample is spun at 3,000 rpm in a microcentrifuge and washed with PBS three times. The pellet is resuspended in PBS. Twenty ml of the resuspended sample is added to a nitrocellulose permeable membrane which has been blocked overnight with 1% BSA blocking compounds and containing immobilized thereon two different antibodies. The first antibody is specific to an antigenic site of the antibodies conjugated with HRP and specific to an antigenic site of E. coli and the second antibody is specific to an antigenic site of the antibodies conjugated with HRP and specific to an antigenic site of Salmonella. The two different antibodies are localized on the permeable membrane in distinct locations to distinguish between the two species of bacteria. The permeable membrane then is washed three times. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where E. coli and Salmonella are attached to the membrane via binding to their respective immobilized antibodies. E. coli and Salmonella then are detected and identified on the permeable membrane due to the color change of the E. coli and Salmonella on the permeable membrane and their respective locations on the permeable membrane, which is distinguished from the color of any other species of bacteria which may be contained in the mixture. E. coli and Salmonella are detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 12

### Rapid Detection and Identification of E. coli and Salmonella Using Antibodies as Reporter and Capture Compounds and Selected Against Two Different Sites of E. coli and Salmonella

A liquid sample containing an unknown number of live bacterial species including the two target bacteria E. coli and Salmonella is set up as described below. Alternatively, a sample containing an unknown mixture of live bacterial species including the two target bacteria E. coli and Salmonella are poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to two different rabbit polyclonal antibodies, one antibody specific for a first antigenic site of E. coli and the other antibody specific for a first antigenic site of Salmonella, is added to bring the final volume up to 20 ml. The 20 ml sample is added to a nitrocellulose permeable membrane which has been blocked overnight with 1% BSA blocking compounds and which has been coated with 20 ml of a 1:40 dilution of two rabbit polyclonal antibodies, the first antibody specific for a second antigenic site of E. coli and the second antibody specific for a second antigenic site of Salmonella. The permeable membrane then is washed in a washing solution (PBS, pH 7.4 with 0.1% Tween 20) using a portable washing device at medium speed of rotation for about two minutes. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where E. coli and Salmonella are attached to the membrane via binding to their respective immobilized antibodies. E. coli and Salmonella then are detected and identified on the permeable membrane due to the color change of the E. coli and Salmonella on the permeable membrane and their respective locations on the permeable membrane, which is distinguished from the color of any other species of bacteria which may be contained in the mixture. E. coli and Salmonella are detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 13

### Rapid Detection and Identification of E. coli and Salmonella Using Aptamers and Antibodies as Reporter Compounds and Aptamers as Capture Compounds

A liquid sample containing an unknown number of live bacterial species including the two target bacteria E. coli and Salmonella is set up as described below. Alternatively, a sample containing an unknown mixture of live bacterial species including the two target bacteria E. coli and Salmonella are poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to aptamers selected against E. coli and antibodies specific for an antigenic site of Salmonella is added to bring the final volume up to 100 ml. The sample is spun at 3,000 rpm in a microcentrifuge and washed with PBS three times. The pellet is resuspended in PBS. Twenty ml of the resuspended sample is added to a nitrocellulose permeable membrane which has been blocked overnight with 1% BSA blocking compounds and containing immobilized thereon two different aptamers (each linked to amine compounds). The first aptamer is selected to bind to the aptamer conjugated with HRP and selected against E. coli and the second aptamer is selected to bind to the antibodies conjugated with HRP and specific for an antigenic site of Salmonella. The two different aptamers are localized on the permeable membrane in distinct locations to distinguish between the two species of bacteria. The permeable membrane then is washed three times. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where E. coli and Salmonella are attached to the membrane via binding to their respective immobilized aptamers. E. coli and Salmonella then are detected and identified on the permeable membrane due to the color change of the E. coli and Salmonella on the permeable membrane and their respective locations on the permeable membrane, which is distinguished from the color of any other species of bacteria which may be contained in the mixture. E. coli and Salmonella are detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 14

### Rapid Detection and Identification of E. coli and Salmonella Using Aptamers and Antibodies as Reporter Compounds and Aptamers as Capture Compounds and Selected Against Two Different Sites of E. coli and Salmonella

A liquid sample containing an unknown number of live bacterial species including the two target bacteria E. coli and Salmonella is set up as described below. Alternatively, a sample containing an unknown mixture of live bacterial species including the two target bacteria E. coli and Salmonella are poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to an aptamer selected for a first antigenic site of E. coli and an antibody specific for a first antigenic site of Salmonella, is added to bring the final volume up to 20 ml. The 20 ml sample is added to a nitrocellulose permeable membrane which has been blocked overnight with 1% BSA blocking compounds and which has been coated with 20 ml of a 1:40 dilution of two aptamers (each linked via an amine compound to the membrane), the first aptamer selected for a second antigenic site of E. coli and the second aptamer selected for a second antigenic site of Salmonella. The permeable membrane then is washed in a washing solution (PBS, pH 7.4 with 0.1% Tween 20) using a portable washing device at medium speed of rotation for about two minutes. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where E. coli and Salmonella are attached to the membrane via binding to their respective immobilized aptamers. E. coli and Salmonella then are detected and identified on the permeable membrane due to the color change of the E. coli and Salmonella on the permeable membrane and their respective locations on the permeable membrane, which is distinguished from the color of any other species of bacteria which may be contained in the mixture. E. coli and Salmonella are detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 15

### Rapid Detection and Identification of E. coli and Salmonella Using Aptamers and Antibodies as Reporter Compounds and Antibodies as Capture Compounds

A liquid sample containing an unknown number of live bacterial species including the two target bacteria E. coli and Salmonella is set up as described below. Alternatively, a sample containing an unknown mixture of live bacterial species including the two target bacteria E. coli and Salmonella are poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to aptamers selected against E. coli and antibodies specific for an antigenic site of Salmonella is added to bring the final volume up to 100 ml. The sample is spun at 3,000 rpm in a microcentrifuge and washed with PBS three times. The pellet is resuspended in PBS. Twenty ml of the resuspended sample is added to a nitrocellulose permeable membrane which has been blocked overnight with 1% BSA blocking compounds and containing immobilized thereon two different antibodies. The first antibody is selected to bind to the aptamers conjugated with HRP and selected against E. coli and the second antibody is selected to bind to the antibodies conjugated with HRP and specific for an antigenic site of Salmonella. The two different antibodies are localized on the permeable membrane in distinct locations to distinguish between the two species of bacteria. The permeable membrane then is washed three times. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where E. coli and Salmonella are attached to the membrane via binding to their respective immobilized antibodies. E. coli and Salmonella then are detected and identified on the permeable membrane due to the color change of the E. coli and Salmonella on the permeable membrane and their respective locations on the permeable membrane, which is distinguished from the color of any other species of bacteria which may be contained in the mixture. E. coli and Salmonella are detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

### Example 16

### Rapid Detection and Identification of E. coli and Salmonella Using Aptamers and Antibodies as Reporter Compounds and Antibodies as Capture Compounds and Selected Against Two Different Sites of E. coli and Salmonella

A liquid sample containing an unknown number of live bacterial species including the two target bacteria E. coli and Salmonella is set up as described below. Alternatively, a sample containing an unknown mixture of live bacterial species including the two target bacteria E. coli and Salmonella are poured on a Petri plate filled with SBA; the plate is incubated at a temperature of about 37° C. for about 3 to 6 hours to grow the bacteria; and, after the bacterial species is grown and microcolonies appear, a sample specimen of the microcolonies is set up as described below. A 0.5 McFarland of the liquid sample or, alternatively, a sample of the grown microcolonies, is prepared as commonly known in the art and the bacteria are serially diluted, starting at about 10⁵ bacteria/ml. To each dilution, a 1:30 dilution of HRP conjugated to an aptamer selected for a first antigenic site of E. coli and an antibody specific for a first antigenic site of Salmonella, is added to bring the final volume up to 20 ml. The 20 ml sample is added to a nitrocellulose permeable membrane which has been blocked overnight with 1% BSA blocking compounds and which has been coated with 20 ml of a 1:40 dilution of two antibodies, the first antibody specific for a second antigenic site of E. coli and the second antibody specific for a second antigenic site of Salmonella. The permeable membrane then is washed in a washing solution (PBS, pH 7.4 with 0.1% Tween 20) using a portable washing device at medium speed of rotation for about two minutes. A drop of DAB (3 mg/ml in Tris saline buffer, pH 7.6), 3% H₂O₂ and 1.2 weight % of NiCl₂ then is added to the permeable membrane. A color reaction to a grey-brown color rapidly develops in about 0.5 to 3 minutes on the surface of the permeable membrane in locations where E. coli and Salmonella are attached to the membrane via binding to their respective immobilized antibodies. E. coli and Salmonella then are detected and identified on the permeable membrane due to the color change of the E. coli and Salmonella on the permeable membrane and their respective locations on the permeable membrane, which is distinguished from the color of any other species of bacteria which may be contained in the mixture. E. coli and Salmonella are detected reliably at 10⁵ to 10² bacteria/ml in as little as 30 minutes, with minimal background observed, and with no cross-reactivity to other species of bacteria. This detection and identification assay is significantly more rapid than any other microbiological diagnostic assay known to date.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1. A method for rapid detection and identification of one or more live target microorganisms in a sample, comprising:
obtaining a sample containing at least one target microorganism;
forming a mixture containing the at least one target microorganism and at least one first aptamer conjugated to a reporter compound and selected for a binding site of the at least one target microorganism and/or a mixture containing at least one first antibody conjugated to a reporter compound and specific for an antigenic site of the at least one target microorganism;
washing the mixture;
adding the mixture to a permeable membrane, said permeable membrane having immobilized thereon at least one second aptamer selected for a binding site of the at least one first aptamer and/or at least one second antibody specific for an antigenic site of the at least one first antibody;
washing any non-bound microorganisms from the permeable membrane; and
adding a detection solution to the permeable membrane in order to detect and identify the at least one target microorganism, wherein the detection and identification of the at least one microorganism is achieved in less than one hour.

2. The method of claim 1, wherein the sample is a liquid sample.

3. The method of claim 1, wherein the sample is obtained by placing a sample containing at least one target microorganism on a nutrient medium and incubating the sample for a period of time in order to grow at least one microcolony from the at least one target microorganism.

4. The method of claim 1, wherein the at least one second aptamer is linked to an amine compound selected from the group consisting of bovine serum albumin, amine linkers and other compounds which contain an amine group, and wherein the at least one first antibody or at least one second antibody is a monoclonal or a polyclonal antibody.

5. The method of claim 1, wherein the reporter compound is selected from the group consisting of chromogenic substrates, fluorogenic substrates and enzymes; and the detection solution is selected from the group consisting of staining solutions such as is 3,3′-diaminobenzidine (DAB) and enzymes such as alkaline phosphatase.

6. The method of claim 5, wherein the reporter compound is the enzyme horse-radish peroxidase and the detection solution is the staining solution 3,3′-diaminobenzidine (DAB).

7. The method of claim 1, wherein the at least one target microorganism is selected from the group consisting of bacteria, yeasts, fungi, spores, eukaryotic cells, cells, viruses and proteins.

8. The method of claim 1, wherein the nutrient medium is an agar selected from the group consisting of tryptic soy agar and sheep blood agar.

9. The method of claim 1, wherein the permeable membrane is selected from the group consisting of polymers such as nitrocellulose, regenerated cellulose and its derivatives such as cellophane, cuprophane and dialysis membranes, nylon or polyvinylidene fluoride (PVDF) and other polymers such as organic or inorganic materials with naturally occurring pores or artificially created pores.

10. A method for rapid detection and identification of one or more live target microorganisms in a sample, comprising:
obtaining a sample containing at least one target microorganism;
forming a mixture containing at least one microcolony from the at least one target microorganism and at least one first aptamer conjugated to a reporter compound and selected for a first binding site of the at least one target microorganism and/or at least one first antibody conjugated to a reporter compound and specific for a first antigenic site of the at least one target microorganism;
adding the mixture to a permeable membrane having immobilized thereon at least one second aptamer selected for a second binding site of the at least one target microorganism and/or at least one second antibody specific for a second antigenic site of the at least one target microorganism;
washing any non-bound microorganisms from the permeable membrane; and
adding a detection solution to the permeable membrane in order to detect and identify the at least one target microorganism, wherein the detection and identification of the at least one microorganism is achieved in less than one hour.

11. The method of claim 10, wherein the sample is a liquid sample.

12. The method of claim 10, wherein the sample is obtained by placing a sample containing at least one target microorganism on a nutrient medium and incubating the sample for a period of time in order to grow at least one microcolony from the at least one target microorganism.

13. The method of claim 10, wherein the at least one second aptamer is linked to an amine compound selected from the group consisting of bovine serum albumin, amine linkers and other compounds which contain an amine group, and wherein the at least one first antibody or at least one second antibody is a monoclonal or a polyclonal antibody.

14. The method of claim 10, wherein the reporter compound is selected from the group consisting of chromogenic substrates, fluorogenic substrates and enzymes; and the detection solution is selected from the group consisting of staining solutions such as is 3,3′-diaminobenzidine (DAB) and enzymes such as alkaline phosphatase.

15. The method of claim 14, wherein the reporter compound is the enzyme horse-radish peroxidase and the detection solution is the staining solution 3,3′-diaminobenzidine (DAB).

16. The method of claim 10, wherein the at least one target microorganism is selected from the group consisting of bacteria, yeasts, fungi, spores, eukaryotic cells, cells, viruses and proteins.

17. The method of claim 10 wherein the nutrient medium is an agar selected from the group consisting of tryptic soy agar and sheep blood agar.

18. The method of claim 10, wherein the permeable membrane is selected from the group consisting of polymers such as nitrocellulose, regenerated cellulose and its derivatives such as cellophane, cuprophane and dialysis membranes, nylon or polyvinylidene fluoride (PVDF) and other polymers such as organic or inorganic materials with naturally occurring pores or artificially created pores.

19. A device for rapid detection and identification of one or more live target microorganisms in a sample, comprising:
a plate having therein nutrient medium to grow at least one microcolony from at least one live target microorganism;
at least one first aptamer conjugated to a reporter compound and selected against a binding site of the at least one target microorganism;
a permeable membrane having immobilized thereon at least one second aptamer selected against a binding site of the at least one first-aptamer;
a portable washing apparatus; and
a detection solution for detecting and identifying the at least one target microorganism bound on the permeable membrane, wherein the device allows for rapid detection and identification of the one or more live target microorganisms in about 30 minutes or less.

20. The device of claim 19, wherein the at least one second aptamer is linked to an amine compound selected from the group consisting of bovine serum albumin, amine linkers and other compounds which contain an amine group.

21. The device of claim 19, wherein the reporter compound is selected from the group consisting of chromogenic substrates, fluorogenic substrates and enzymes and the detection solution is selected from the group consisting of 3,3′-diaminobenzidine (DAB) staining solutions and alkaline phosphatase enzymes.

22. The device of claim 21, wherein the reporter compound is the enzyme horse-radish peroxidase and the detection solution is the staining solution DAB.

23. The device of claim 19, wherein at least one target microorganism is selected from the group consisting of bacteria, yeasts, fungi, spores, eukaryotic cells, cells, viruses and proteins.

24. The device of claim 19, wherein the nutrient medium is an agar selected from the group consisting of tryptic soy agar and sheep blood agar.

25. The device of claim 19, wherein the permeable membrane is selected from the group consisting of nitrocellulose, regenerated cellulose, cellophane, cuprophane, dialysis membranes, nylon, polyvinylidene fluoride (PVDF) and other organic or inorganic polymer materials with naturally occurring pores or artificially created pores.

26. A device for rapid detection and identification of one or more live target microorganisms in a sample, comprising:
a plate having therein nutrient medium to grow at least one microcolony from at least one live target microorganism;
at least one first aptamer conjugated to a reporter compound and selected against a first binding site of the at least one target microorganism;
a permeable membrane having immobilized thereon at least one second aptamer selected against a second binding site of the at least one target microorganism;
a portable washing apparatus; and
a detection solution for detecting and identifying the at least one target microorganism bound on the permeable membrane, wherein the device allows for rapid detection and identification of the one or more live target microorganisms in about 30 minutes or less.

27. The device of claim 26, wherein the at least one second aptamer is linked to an amine compound selected from the group consisting of bovine serum albumin, amine linkers and other compounds which contain an amine group.

28. The device of claim 26, wherein the reporter compound is selected from the group consisting of chromogenic substrates, fluorogenic substrates and enzymes and the detection solution is selected from the group consisting of 3,3′-diaminobenzidine (DAB) staining solutions and alkaline phosphatase enzymes.

29. The device of claim 28, wherein the reporter compound is the enzyme horse-radish peroxidase and the detection solution is the staining solution DAB.

30. The device of claim 26, wherein at least one target microorganism is selected from the group consisting of bacteria, yeasts, fungi, spores, eukaryotic cells, cells, viruses and proteins.

31. The device of claim 26, wherein the nutrient medium is an agar selected from the group consisting of tryptic soy agar and sheep blood agar.

32. The device of claim 26, wherein the permeable membrane is selected from the group consisting of nitrocellulose, regenerated cellulose, cellophane, cuprophane, dialysis membranes, nylon, polyvinylidene fluoride (PVDF) and other organic or inorganic polymer materials with naturally occurring pores or artificially created pores.

33. A test kit for rapid detection and identification of one or more live target microorganisms in a sample, comprising:
a plate having therein nutrient medium to grow at least one microcolony from at least one live target microorganism;
at least one first-aptamer conjugated to a reporter compound and selected against a binding site of the at least one target microorganism;
a permeable membrane having immobilized thereon at least one second aptamer selected against a binding site of the at least one first-aptamer;
a portable washing apparatus; and
a detection solution for detecting and identifying the at least one target microorganism bound on the permeable membrane, wherein the test kit allows for rapid detection and identification of the one or more live target microorganisms in about 30 minutes or less.

34. A test kit for rapid detection and identification of one or more live target microorganisms in a sample, comprising:
a plate having therein nutrient medium to grow at least one microcolony from at least one live target microorganism;
at least one first aptamer conjugated to a reporter compound and selected against a first binding site of the at least one target microorganism;
a permeable membrane having immobilized thereon at least one second aptamer selected against a second binding site of the at least one target microorganism;
a portable washing apparatus; and
a detection solution for detecting and identifying the at least one target microorganism bound on the permeable membrane in, wherein the test kit allows for rapid detection and identification of the one or more live target microorganisms in about 30 minutes or less.
